(19) United States
(12) Patent Application Publication
(10) Pub. No.: US 2023/0397824 A1
(43) Pub. Date: Dec. 14, 2023

(54) CONTACTLESS BREATHING OR HEARTBEAT DETECTION METHOD

(71) Applicant: PONTOSENSE INC., Waterloo (CA)

(72) Inventors: Yang YANG, Guangdong (CN); Yunlong LUO, Guangdong (CN); Alex QI, Waterloo (CA); Ge SHI, Guangdong (CN); Yihong QI, Guangdong (CN); Ruini XUE, Guangdong (CN); Ronghao JIN, Guangdong (CN)

(21) Appl. No.: 18/251,392

(22) PCT Filed: Nov. 3, 2021
(86) PCT No.: PCT/CN2021/128302

§ 371 Date: May 1, 2023

(30) Foreign Application Priority Data
Nov. 4, 2020 (CN) 202011218737.2

(51) Int. Cl. A61B 5/0205; A61B 5/00

(52) U.S. Cl. CPC A61B 5/0205; A61B 5/725; A61B 5/0022; A61B 5/7225

(57) ABSTRACT

The invention relates to the technical field of wireless sensing, in particular to a non-contact breathing or heartbeat detection method, which comprises the following steps: S1, acquiring channel state information of a wireless signal according to an output wireless signal and a wireless signal reflected by a measured target; S2, extracting a vital sign waveform signal from the channel state information of the wireless signal; S3, performing filtering on the vital sign waveform signal based on Huber-Kalman filtering algorithm to obtain the filtered vital sign waveform signal; S4, extracting vital sign parameters from the filtered vital sign waveform signal. In the method, the Huber objective function is used to improve the classical Kalman filtering algorithm, and the Huber-Kalman filtering algorithm is used for filtering processing, so that the human vital sign detection method is more robust, and the extracted vital sign parameters can reflect the human vital signs more accurately.

### TECHNICAL FIELD

The invention relates to the technical field of wireless sensing, in particular to a contactless breathing or heartbeat detection method.

### BACKGROUND

With the transformation of life style in human society and the development of science and technology, people pay more attention to health and have a strong interest in the detection of ubiquitous vital signs. Traditional vital signs monitoring methods require wearing special instruments, such as bracelets or pulse oximeters. These technologies are inconvenient and uncomfortable to use. The contactless breathing and heartbeat detection scheme based on Wi-Fi wireless sensing, non-contact, easy to deploy and low-cost long-term vital signs monitoring is very attractive. Contactless breathing and heartbeat detection can be widely used in family scenes and car scenes, and can effectively detect the breathing and heartbeat of the measured target.

In the prior art, based on the concept of Fresnel zone, the amplitude information of CSI is usually used for a contactless breathing and heartbeat detection. As the closest prior art of the invention, the invention patent, a method for detecting human respiration based on Wi-Fi channel state signals (publication number CN109998549A), provides a solution. A channel state signal data acquisition platform is built through symmetrically arranged Wi-Fi access points and monitoring points, and a Fresnel field is established. The human body is located between the Wi-Fi access points and the monitoring points. Based on the Hampel filtering algorithm, the outliers are filtered out, and the subcarrier with the largest variance is selected. The CSI signal of the selected subcarrier is decomposed into components at different scales by using multi-resolution discrete wavelet transform, from which the human respiratory frequency is extracted.

Although this technology has realized the detection of human respiratory rate and its deployment is simple, there are still some problems. For example, in the above scheme, only the commonly used filtering algorithm, such as Hampel algorithm, is used to filter out abnormal values. In the filtering stage, environmental factors and the distribution of errors are not considered, which will cause that small errors can only be filtered out, and the filtered subcarrier signals will still have interference. Taking the automobile usage scene as an example, using Wi-Fi signals to detect vital signs will be affected by dynamic noise in the environment. For example, when a car turns a corner, it will bring big errors when it passes through the speed bump. These big errors can be filtered out by the conventional filtering algorithm in the detection of vital signs such as breathing and heartbeat. However, the engine jitter or the tiny jitter caused by the car passing through the gravel road always exists, and the filtering effect of such small errors by the conventional filtering algorithm is not good, which makes the extracted vital signs signals inaccurate. Therefore, the scheme in the prior art has high requirements on the environment, can not avoid the introduction of small errors, and can not adapt to various practical application scenarios.

### SUMMARY

The purpose of the invention is to overcome the problem that the conventional filtering algorithm mentioned above cannot filter out tiny errors, improve the classical Kalman filtering algorithm, replace the second norm in the Kalman filtering algorithm with a Huber objective function (including the first norm and the second norm), balance the big error and the small error, and improve the classical Kalman filtering algorithm by using the Huber objective function, so that the human breathing and heartbeat detection method based on Wi-Fi channel state information is more robust and can be applied to various scenarios. Therefore, a contactless breathing or heartbeat detection method is proposed.

In order to achieve the above object, the present invention provides the following technical scheme:

A contactless breathing or heartbeat detection method, including the following steps:
S1, acquiring channel state information of a wireless signal according to an outputted wireless signal and a wireless signal reflected back by a detected target;
S2, extracting a vital sign waveform signal from the channel state information of the wireless signal;
S3, performing filtering on the vital sign waveform signal on the basis of a Huber-Kalman filtering algorithm to obtain a filtered vital sign waveform signal, and the Huber-Kalman filtering algorithm uses the Huber objective function to update the formula of the Kalman filtering algorithm;
S4, extracting respiratory characteristic parameters and/or heartbeat characteristic parameters from the filtered vital sign waveform signals.

As a preferred scheme of the present invention, in step S3, the Huber-Kalman filtering algorithm uses the Huber objective function to update the formula of the Kalman filtering algorithm, specifically including the following steps:

The iterative calculation process of Kalman equation is modified by Huber objective function, and the input vital sign waveform signal is filtered.

Huber objective function divides the error into two parts, including big error and small error. Big error refers to the error value that deviates from the real value and is greater than the big error threshold, and small error refers to the error value that fluctuates within the small error threshold based on the real value.

Iterative calculation means that the optimal estimated value at the current moment is determined by the optimal estimated value calculated according to Kalman update equation at the previous moment and the observed value calculated according to Huber objective function at the current moment.

As a preferred scheme of the present invention, in step S3, the prediction equation based on Huber objective function is expressed as:

$${\hat{x}}_{k}^{-} = A {\hat{x}}_{k - 1} + B {u}_{k - 1}$$

$${ρ}_{a}^{-} ( {e}_{k + 1}^{-} ) = \{ \begin{matrix}{A}^{2} {ρ}_{a} ( {e}_{k} ) + \frac{Q}{2} & if 0 \leq {ρ}_{a} ( {e}_{k} ) \leq \frac{{a}^{2}}{2} \\ \vert A \vert ( {ρ}_{a} ( {e}_{k} ) + \frac{{a}^{2}}{2} ) - \frac{{a}^{2}}{2} & if {ρ}_{a} ( {e}_{k} ) > \frac{{a}^{2}}{2}\end{matrix}$$

Kalman update equation is:

$${K}_{k} = \{ \begin{matrix}\frac{2 {ρ}_{a}^{-} ( {e}_{k}^{-} ) H}{2 {ρ}_{a}^{-} ( {e}_{k}^{-} ) {H}^{2} + R} & if 0 \leq {ρ}_{a}^{-} ( {e}_{k}^{-} ) \leq \frac{{a}^{2}}{2} \\ \frac{1}{H} & if {ρ}_{a}^{-} ( {e}_{k}^{-} ) > \frac{{a}^{2}}{2}\end{matrix}$$

$${\hat{x}}_{k} = {\hat{x}}_{k}^{-} + {K}_{k} ( {z}_{k} - H {\hat{x}}_{k}^{-} )$$

$${ρ}_{a} ( {e}_{k} ) = \{ \begin{matrix}{ρ}_{a}^{-} ( {e}_{k}^{-} ) ( 1 + {K}_{k}^{2} {H}^{2} - 2 {K}_{k} H ) + \frac{1}{2} {K}_{k}^{2} R & if 0 \leq {ρ}_{a}^{-} ( {e}_{k}^{-} ) \leq \frac{{a}^{2}}{1 - {K}_{k} H} - \frac{{a}^{2}}{2} \\ \vert 1 - {K}_{k} H \vert ( {ρ}_{a}^{-} ( {e}_{k}^{-} ) + \frac{{a}^{2}}{2} ) - \frac{{a}^{2}}{2} & if {ρ}_{a}^{-} ( {e}_{k}^{-} ) > \frac{{a}^{2}}{1 - {K}_{k} H} - \frac{{a}^{2}}{2}\end{matrix}$$

Where k represents the kth moment; a represents the threshold between a big error and a small error; \hat{x}ₖ⁻ represents the predicted value at moment k, and \hat{x}ₖ₋₁ represents the optimal estimated value at moment k−1; zₖ is the input data; uₖ₋₁ represents the random noise in the state transition process; vₖ represents measurement noise; Q represents the covariance of process noise; R represents the measurement noise covariance; A represents the state transition coefficient; B represents the control input coefficient; H represents the measurement coefficient; eₖ stands for posterior error; eₖ⁻ represents prior error; ρₐ⁻ (eₖ⁻) represents a prior error function; ρₐ(eₖ) represents a posterior error function; Kₖ stands for Kalman gain.

As a preferred scheme of the present invention, step S3 further includes detecting the environmental noise level in real time, and adjusting the threshold between the big error and the small error according to the environmental noise level.

As a preferred scheme of the present invention, step S4 specifically includes the following steps:
A41, segmenting the filtered vital sign waveform signal according to the time window to obtain a vital sign waveform;
A42, extracting the time interval between the peaks of the vital sign waveform, and determining the frequency of breathing or heartbeat of the tested human body according to the time interval between the peaks.

As a preferred scheme of the present invention, step S4 specifically includes the following steps:
B41, segmenting the filtered vital sign waveform signal according to the time window to obtain a vital sign waveform;
B42, performing frequency domain analysis on the vital sign waveform to obtain the frequency spectrum characteristics of the vital sign waveform;
B43, performing low-pass filtering on the spectrum characteristics of the vital sign waveform to obtain the breathing frequency of the tested human body and/or performing high-pass filtering on the spectrum characteristics of the vital sign waveform to obtain the heartbeat frequency of the tested human body.

As a preferred scheme of the present invention, when the wireless signal is a millimeter wave radar signal, step S1 specifically includes the following steps:
S11, outputting a millimeter wave radar signal to a measured target, and taking the millimeter-wave radar signal at the transmitting time as a reference signal;
S12, the measured target reflects the millimeter wave radar signal to form an echo signal, and the echo signal and the reference signal are demodulated to generate an intermediate frequency signal;
S13, performing ADC sampling and FFT transformation on the intermediate frequency signal in turn to obtain the distance information and the phase information of the measured target;
S14, the phase information of the millimeter wave radar signal is used as the channel state information of the millimeter wave radar signal.

As a preferred scheme of the present invention, the range of the frequency f of the millimeter wave radar signal includes: 23 GHz≤F≤28 GHz, 60 GHz≤F≤65 GHz and 76 GHz≤F≤81 GHz.

As a preferred scheme of the present invention, when the wireless signal is a Wi-Fi signal, step S1 specifically includes the following steps:
C11, outputting the wireless signal to the measured target, and simultaneously using the wireless signal as a reference signal, and transmitting the wireless signal in a wired way;
C12: The measured object reflects the wireless signal to form a reflected wireless signal, and the reflected wireless signal is differentiated from the reference signal to obtain the phase difference information of the wireless signal, which is used as the channel state information of the wireless signal.

As a preferred scheme of the present invention, step S2 specifically includes the following steps:
S21, unwrapping the phase information to obtain a preprocessed signal;
S22, performing subcarrier fusion processing on the preprocessed signal to output a respiratory characteristic waveform signal.

As a preferred scheme of the present invention, when the wireless signal is a Wi-Fi signal in 2.4G band, the frequency bandwidth of the channel state information is 20 MHz or 40 MHz, and the frequency range of the subcarrier signal of the channel state information is 2401 MHz to 2483 MHz;

When the wireless signal is a Wi-Fi signal in 5G band, the frequency bandwidth of the channel state information is 20 MHz, 40 MHz or 80 MHz, and the frequency range of the subcarrier signal of the channel state information is 5150 MHz to 5850 MHz.

Based on the same idea, the invention also provides a contactless breathing or heartbeat detection system, which comprises a wireless signal transmitting device, a wireless signal receiving device and a data processor.

The wireless signal transmitting device outputs a wireless signal to a measured target;

The wireless signal receiving device receives the wireless signal reflected by the measured target;

The data processor executes any one of the above-mentioned contactless vital sign detection methods according to the wireless signal output by the wireless signal transmitter and the wireless signal reflected by the measured target, and calculates the respiratory characteristic parameters and/or heartbeat characteristic parameters of the measured target.

As a preferred scheme of the present invention, the wireless signal transmitting device comprises a wireless signal generating element and a transmitting antenna, and the wireless signal receiving device comprises a receiving antenna and a wireless signal receiving element;

The wireless signal generating element radiates the generated wireless signal to the measured target through the transmitting antenna;

The wireless signal receiving element receives the wireless signal reflected by the measured target through the receiving antenna;

The transmitting antenna and the receiving antenna are circularly polarized, and the polarization directions of the transmitting antenna and the receiving antenna are opposite.

As a preferred scheme of the invention, the clock signal on which the wireless signal generating element generates the wireless signal is the same as the clock signal on which the data processor receives the wireless signal reflected by the measured object.

As a preferred scheme of the present invention, when the wireless signal is a Wi-Fi signal, the system further comprises a power distributor,

The Wi-Fi signal generating element outputs the generated Wi-Fi signal to a power distributor,

The power distributor outputs the received Wi-Fi signal to the transmitting antenna and simultaneously outputs the Wi-Fi signal to the data processor through the coaxial cable;

The data processor generates the channel state information of the Wi-Fi signal according to the Wi-Fi signal received from the coaxial cable and the Wi-Fi signal reflected by the measured object.

The beneficial effects of the invention and its preferred scheme are as follows:
1. In the method of the present invention, the Huber objective function is used to improve the classical Kalman filtering algorithm, and the Huber-Kalman filtering algorithm is constructed, and the CSI signal is filtered by the Huber-Kalman filtering algorithm, so that the method for detecting the human vital signs of the channel state signal is more robust, and can be applied to various scenarios, and the vital signs parameters extracted from the filtered CSI signal can reflect the human vital signs more accurately.
2. Through the improved Huber-Kalman filtering algorithm, the formula in the Kalman filtering algorithm is updated by using the Huber objective function including the first norm and the second norm, so that both the big error and the small error can be taken into account in the algorithm. On the one hand, the small error is persistent fluctuation (for example, the continuous jitter of the vehicle), and the small error is filtered by the second norm; On the other hand, the big error is the occasional fluctuation (such as the jitter when the vehicle passes through the speed bump), and the big error is filtered by the first norm. The significance of Huber-Kalman algorithm is to deal with all kinds of big errors and small errors more comprehensively and delicately, so that the filtered signals can reflect the characteristics of breathing and heartbeat more accurately.
3. In the method of the present invention, the degree of environmental noise is detected in real time, and the threshold between big error and small error is adjusted in real time according to the degree of environmental noise. When the Huber-Kalman filtering algorithm is used to filter CSI signals, the filtering range can be dynamically adjusted according to the proportion of big error and small error in the environment, thus improving the environmental adaptability of the method of the present invention.
4. The invention provides a variety of methods for extracting heartbeat or respiratory frequency after filtering, including calculating the frequency of vital signs according to the number of detected peaks in unit time, calculating the frequency of vital signs according to the time interval between peaks, and filtering the channel state signal after frequency domain analysis to obtain the frequency of vital signs.
5. In the process of obtaining channel state information in the present invention, according to the types of wireless signals, the present invention provides two methods: first, the same wireless signal is divided into two identical signals, one for output to the measured target and the other for wired transmission as a reference signal. This method is mainly aimed at wireless signals similar to Wi-Fi signals, and the phase information of such signals is random, so the phase information itself has no clear meaning, so it is necessary to obtain a reference signal. Secondly, the output wireless signal is radiated to the measured target, and the phase information of the reflected signal passing through the reflecting surface can be obtained. This method is mainly aimed at the signal similar to millimeter wave radar.
6. Based on the same idea, the invention also discloses a non-contact breathing or heartbeat detection system, which comprises a wireless signal transmitting device, a wireless signal receiving device and a data processor. In order to prevent multipath interference, when collecting data, the transmitting antenna and the receiving antenna are circularly polarized, and the polarization directions of the transmitting antenna and the receiving antenna are opposite.
7. Further, the clock signal on which the wireless signal generating element generates the wireless signal is the same as the clock signal on which the data processor receives the wireless signal. The whole system works under the same clock signal, which realizes the synchronization of data processing and avoids the system error caused by the unsynchronized equipment clock.
8. Further, if the wireless signal is a Wi-Fi signal, in a contactless vital sign detection system, a power divider and a coaxial cable are used to transmit the reference phase signal, so that the reference phase signal is relatively stable in the phase difference calculation process, and errors caused by the deviation of the reference phase signal are avoided from being introduced into the channel state signal of the Wi-Fi signal.

### BRIEF DESCRIPTION OF THE DRAWINGS

FIG. 1 is a flowchart of a contactless breathing or heartbeat detection method in Embodiment 1 of the present invention;

FIG. 2 is a waveform signal diagram of vital signs used for extracting respiration or heartbeat parameters during the driving of the automobile in Embodiment 1 of the present invention;

FIG. 3 is an effect diagram of separation of respiration and heartbeat in Embodiment 1 of the present invention;

FIG. 4 is a trend diagram of the proportion of small errors in Embodiment 1 of the present invention;

FIG. 5 is an unwrapped respiratory waveform diagram in Embodiment 1 of the present invention;

FIG. 6 is a respiratory waveform diagram after unwinding in Embodiment 1 of the present invention;

FIG. 7 is a waveform diagram of No. 50 subcarrier in Embodiment 1 of the present invention;

FIG. 8 is a waveform diagram of No. 90 subcarrier in Embodiment 1 of the present invention;

FIG. 9 is a schematic diagram of the radar system data acquisition process in Embodiment 2 of the present invention;

FIG. 10 is a flowchart of vital sign detection software in embodiment 2 of the present invention;

FIG. 11 is a structural diagram of a contactless breathing or heartbeat detection system in Embodiment 3 of the present invention;

FIG. 12 is a structural diagram of a contactless breathing or heartbeat detection system including a Wi-Fi signal generating element, a transmitting antenna, a Wi-Fi signal receiving element and a receiving antenna in Embodiment 3 of the present invention;

FIG. 13 is a structural diagram of a contactless breathing or heartbeat detection system including a power distributor in Embodiment 3 of the present invention.

### DETAILED DESCRIPTION

In the following, the invention will be further described in detail in combination with experimental examples and specific embodiments. However, it should not be understood that the scope of the above-mentioned subject matter of the present invention is limited to the following embodiments, and all technologies realized based on the contents of the present invention belong to the scope of the present invention.

### Embodiment 1

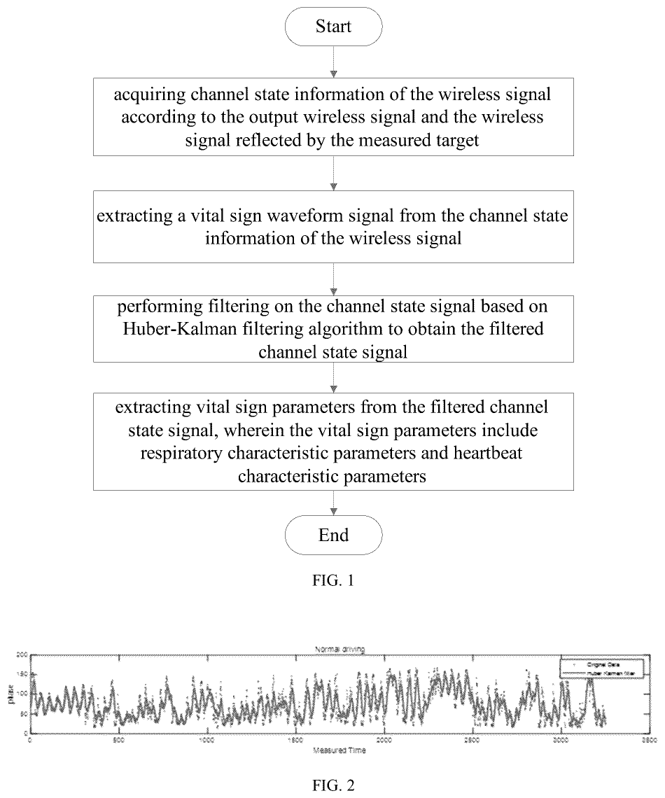

This embodiment discloses a contactless breathing or heartbeat detection method, the flow chart of which is shown in FIG. 1, including the following steps:

S1, acquiring channel state information of a wireless signal according to an outputted wireless signal and a wireless signal reflected back by a detected target.

S2, extracting a vital sign waveform signal from the channel state information of the wireless signal.

S3, performing filtering on the vital sign waveform signal on the basis of a Huber-Kalman filtering algorithm to obtain a filtered vital sign waveform signal, and the Huber-Kalman filtering algorithm uses the Huber objective function to update the formula of the Kalman filtering algorithm;

S4, extracting vital sign parameters from the filtered vital sign waveform signals, wherein the vital sign parameters include respiratory characteristic parameters and/or heartbeat characteristic parameters.

As a preferred scheme, in step S4, vital sign parameters are extracted from the filtered vital sign waveform signal, and the extracted vital sign parameters include respiration rate, respiration times, heartbeat times, heartbeat rate and the like. As a specific embodiment, when the method of the present invention is used in an automobile scene, the waveform signal of vital signs used to extract breathing or heartbeat parameters during the driving of the automobile is shown in FIG. 2. The method for extracting the number of breathing and heartbeats is to calculate the number of peak values of vital sign waveforms within a preset period of time, and calculate the number of breaths or heartbeats through the peak values of vital sign waveforms, specifically including the following steps:
A31, segmenting the filtered vital sign waveform signal according to the time window to obtain a vital sign waveform;
A32, within a preset period of time, calculating the number of peak values of vital sign waveforms, and determining the number of breathing or heartbeats of the tested human body according to the number of peak values of vital sign waveforms.

The method of extracting respiration rate or heartbeat rate can also be to calculate the time interval between the peak values of vital sign waveforms, and calculate respiration rate or heartbeat rate through the time interval.

For the extraction of respiration rate or heartbeat rate, another method can be used:

Firstly, the filtered vital sign waveform signal is analyzed in frequency domain (for example, fast Fourier transform (FFT)), that is, the vital sign waveform signal is transformed from a time domain signal to a frequency domain signal to obtain the frequency spectrum of the filtered vital sign waveform signal.

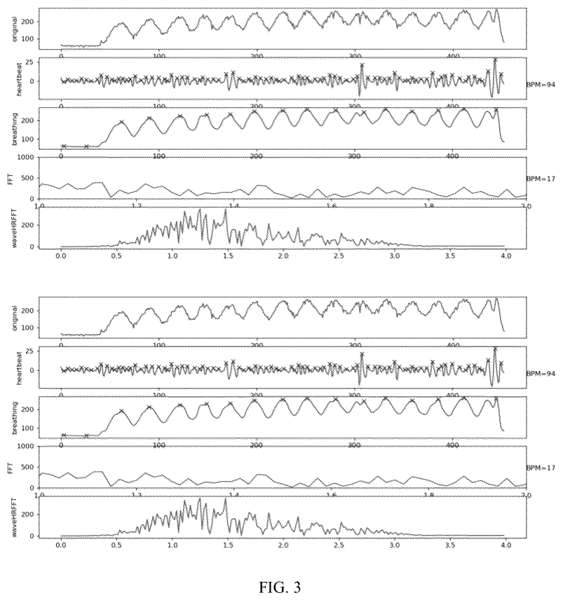

The effect diagram of breathing and heartbeat separation is shown in FIG. 3. BPM=94 is the frequency of heartbeat calculated by counting the peak values in the waveform diagram, and BPM=17 is the respiratory frequency calculated by counting the peak values in the waveform diagram.

As a preferred scheme, in step S3, the vital sign waveform signal is filtered based on the Huber-Kalman filtering algorithm to filter out interference and obtain an accurate filtered vital sign waveform signal. The Huber-Kalman filtering algorithm uses the advantage that the first norm and the second norm can be fused in the Huber objective function to improve the Kalman filtering algorithm. The specific steps include: when calculating the optimal estimated value at the current moment according to the Kalman update equation, the optimal estimated value is determined by the optimal estimated value at the previous moment and the observed value calculated according to the Huber prediction equation at the current moment, and the input vital sign waveform signal is filtered through repeated iterative calculation of the prediction equation and the update equation. In classical Kalman filtering, the Kalman gain is determined by the second norm (that is, the least square method). When there is a big deviation between the measured value and the real value, the result of classical Kalman filtering will be biased towards the error point, and the filtering effect is poor. Based on Huber's objective function, the error is divided into big error and small error, wherein big error refers to the error point that deviates from the real value more than a certain threshold, and small error refers to the error point that fluctuates up and down around the real value in a certain small range (within a certain threshold). Dealing with different types of errors in sections can effectively restore the original breathing and heartbeat waveforms.

In Huber-Kalman filtering algorithm, the prediction equation based on Huber objective function is expressed as:

$${\hat{x}}_{k}^{-} = A {\hat{x}}_{k - 1} + B {u}_{k - 1}$$

$${ρ}_{a}^{-} ( {e}_{k + 1}^{-} ) = \{ \begin{matrix}{A}^{2} {ρ}_{a} ( {e}_{k} ) + \frac{Q}{2} & if 0 \leq {ρ}_{a} ( {e}_{k} ) \leq \frac{{a}^{2}}{2} \\ \vert A \vert ( {ρ}_{a} ( {e}_{k} ) + \frac{{a}^{2}}{2} ) - \frac{{a}^{2}}{2} & if {ρ}_{a} ( {e}_{k} ) > \frac{{a}^{2}}{2}\end{matrix}$$

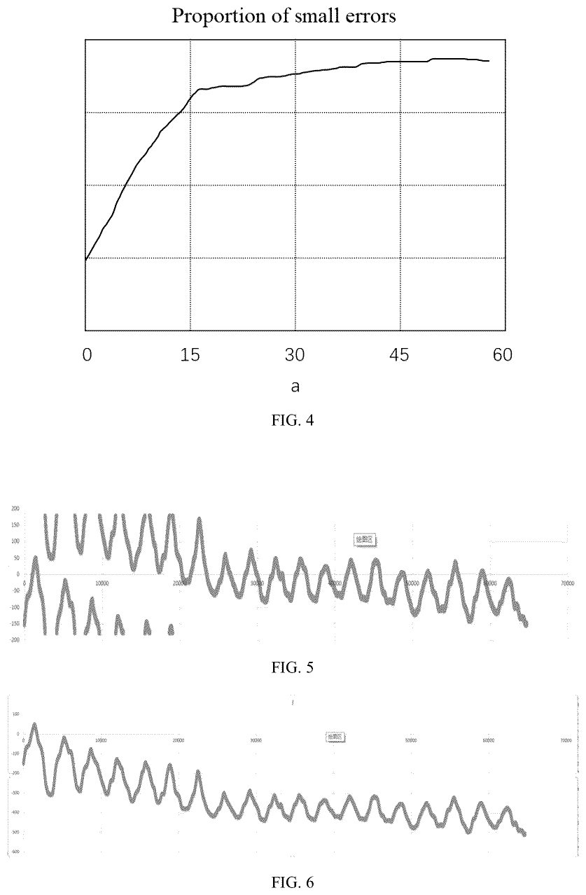

Kalman update equation is:

$${K}_{k} = \{ \begin{matrix}\frac{2 {ρ}_{a}^{-} ( {e}_{k}^{-} ) H}{2 {ρ}_{a}^{-} ( {e}_{k}^{-} ) {H}^{2} + R} & if 0 \leq {ρ}_{a}^{-} ( {e}_{k}^{-} ) \leq \frac{{a}^{2}}{2} \\ \frac{1}{H} & if {ρ}_{a}^{-} ( {e}_{k}^{-} ) > \frac{{a}^{2}}{2}\end{matrix}$$

$${\hat{x}}_{k} = {\hat{x}}_{k}^{-} + {K}_{k} ( {z}_{k} - H {\hat{x}}_{k}^{-} )$$

$${ρ}_{a} ( {e}_{k} ) = \{ \begin{matrix}{ρ}_{a}^{-} ( {e}_{k}^{-} ) ( 1 + {K}_{k}^{2} {H}^{2} - 2 {K}_{k} H ) + \frac{1}{2} {K}_{k}^{2} R & if 0 \leq {ρ}_{a}^{-} ( {e}_{k}^{-} ) \leq \frac{{a}^{2}}{1 - {K}_{k} H} - \frac{{a}^{2}}{2} \\ \vert 1 - {K}_{k} H \vert ( {ρ}_{a}^{-} ( {e}_{k}^{-} ) + \frac{{a}^{2}}{2} ) - \frac{{a}^{2}}{2} & if {ρ}_{a}^{-} ( {e}_{k}^{-} ) > \frac{{a}^{2}}{1 - {K}_{k} H} - \frac{{a}^{2}}{2}\end{matrix}$$

Where k represents the kth moment; a represents the threshold between a big error and a small error; \hat{x}ₖ⁻ represents the predicted value at moment k, and \hat{x}ₖ₋₁ represents the optimal estimated value at moment k−1; zₖ is the input data; uₖ₋₁ represents the random noise in the state transition process; vₖ represents measurement noise; Q represents the covariance of process noise; R represents the measurement noise covariance; A represents the state transition coefficient; B represents the control input coefficient; H represents the measurement coefficient; eₖ stands for posterior error; eₖ⁻ represents prior error; ρₐ⁻ (eₖ⁻) represents a prior error function; pₐ(eₖ) represents a posterior error function; Kₖ stands for Kalman gain.

Wherein, the threshold a between big error and small error is used to determine the proportion of big error and small error in filtering. The selection of threshold a is related to the current scene, and the value of acquisition parameter A is different in different scenes. As a preferred scheme, the characteristic value of the environment is detected in real time to determine the environmental state of the tested human body, and the threshold between the big error and the small error is adjusted in real time according to the characteristic value of the environment. For example, in the scene of car driving, the human body, as the reflection surface of Wi-Fi signals, has different relative distances from the signals transmitted by Wi-Fi through different states, including normal driving state, fast starting state and braking state. Detect the characteristic values of the environment (such as the relative distance between the human body and the Wi-Fi signal) to judge the driving scene of the car, and determine the value of the parameter a according to the state of the measured human body (normal driving state, quick start state, braking state, etc.), and adjust the proportion of big errors or small errors in real time. The characteristic value of the detection environment can also be environmental noise, and the state of the detected human body can be judged according to the environmental noise (the normal driving state, the quick start state, the braking state, etc. can be determined by the characteristics of the noise signal). As a specific example, in the case of high-speed driving, the trend chart of small error ratio is shown in FIG. 4. Before a=15, the proportion of small errors is increasing, and after a>15, it tends to be flat. At this time, the inflection point a=15 is selected as the threshold to distinguish big errors from small errors, and Huber-Kalman filtering is performed.

In step S1, according to the difference of wireless signals, the acquisition ways of channel status information are different. In this embodiment, the wireless signal is described as a Wi-Fi signal, but it is not limited that only the Wi-Fi signal can be used. Based on the wireless signal, the same principle and steps are adopted, which is also within the protection scope of the present invention.

As a preferred scheme, when the wireless signal is a Wi-Fi signal, step S1 specifically includes the following steps:
S11, dividing the output Wi-Fi signal into two identical Wi-Fi signals, a first Wi-Fi signal and a second Wi-Fi signal, wherein the first Wi-Fi signal is output near a human body, and the second Wi-Fi signal is used as a reference Wi-Fi signal;
S12, the first Wi-Fi signal is reflected by the human body to form a Wi-Fi signal reflected by the human body, and the difference between the Wi-Fi signal reflected by the human body and the reference Wi-Fi signal is obtained, and the phase difference information of the Wi-Fi signal is used as the channel state information of the Wi-Fi signal.

As a preferred scheme, when the vital signs extracted in step S4 are respiratory characteristic parameters, step S2 performs subcarrier fusion on the channel state information of the wireless signal to obtain the vital signs waveform signal, which specifically includes the following steps:

S21, unwrapping the phase difference signal to obtain a preprocessed signal. To calculate the phase-frequency characteristics, it is necessary to use the arc tangent function. The arc tangent function in the computer stipulates that the angle in the first and second quadrants is 0˜π, and the angle in the third and fourth quadrants is 0˜−π. If an angle changes from 0 to 2π, but the actual result is 0˜π, and then from −π to 0, a jump occurs at w=π, and the jump amplitude is 2π, which is called phase winding. In python and MATLAB, unwrap (w) is the unwrapping function, which makes the phase not jump at π, thus reflecting the real phase change. The unwrapped respiratory waveform is shown in FIG. 5, and the obtained waveform is discontinuous due to the jump at w=π. The unwrapped respiratory waveform is shown in FIG. 6, which reflects the real phase change because the phase does not jump at π, and the unwrapped respiratory waveform is continuous, which is convenient for subsequent peak extraction.

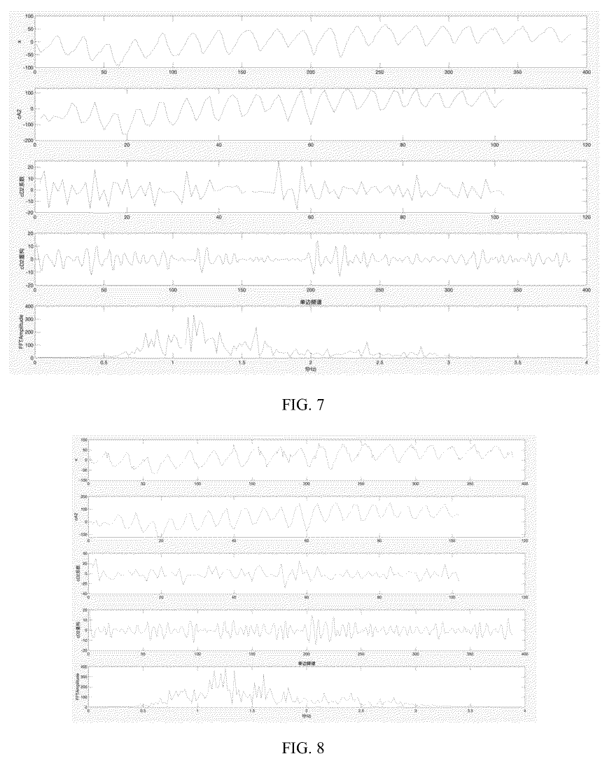

S22, performing subcarrier fusion processing on the preprocessed signal to output respiratory characteristic waveform signals. In Wi-Fi wireless sensing, CSI has 53 subchannels, and each subchannel has multiple subcarriers. Because of the different center frequency of each subcarrier, each subcarrier has different sensitivity to motion at different speeds. By selecting multiple subcarriers to complement each other, the characteristics of respiratory waveform are reflected. FIG. 7 shows the waveform diagram of No. 50 subcarrier and FIG. 8 shows the waveform diagram of No. 90 subcarrier. The characteristics of these two waveforms are not exactly the same. Therefore, by superimposing these two subcarriers, the signal complementation is realized and the integrity of the extracted vital signs is ensured.

Step S22 specifically includes the following steps:

S221: Obtain the subcarrier signal of each channel state information in the preprocessed signal, and the frequency of the subcarrier signal is distributed in the frequency bandwidth of the channel state information.

S222: In each channel state information, some subcarrier signals are extracted at intervals of N frequency points to form a preselected subcarrier signal. For example, the subcarrier signal is extracted every 1 frequency point, every 2 frequency points, every 3 frequency points, and how many frequency points are separated from each other to calculate the demand determination.

S223: Calculate the weight value and absolute deviation value corresponding to the preselected subcarrier signal.

S224: Multiply the weight values corresponding to the pre-selected subcarrier signals with the absolute deviation values to calculate the correction data of each pre-selected subcarrier signal, and output the vital sign waveform signal after superimposing the correction data.

In step S223, the original data of the sub-carriers of the pre-selected sub-carrier signal is set to X₁={x₁₁, x₁₂, . . . , x₁ₙ}, X₂={x₂₁, x₂₂, . . . , x₂ₙ}, Xₘ={xₘ₁, xₘ₂, . . . , xₘₙ}, that is, the absolute deviation value of each sub-carrier can be calculated separately. The calculation formula is:

$${M}_{m} = \frac{1}{n} {\sum }_{i = 1}^{n} \vert {x}_{m i} - {\overline{x}}_{m} \vert$$

Wherein, n is the sampling number of each preselected subcarrier signal after discrete processing, m is the number of preselected subcarrier signals, xₘᵢ is the sampling value of each preselected subcarrier signal after discrete processing, and x̅ₘ is the average value of the sampling values in the mth preselected subcarrier signal.

The formula for calculating the corresponding weight of each subcarrier is:

$${W}_{m} = \frac{{M}_{m}}{{{\sum }_{i = 1}}^{m} {M}_{i}}$$

Therefore, in step S224, the result of subcarrier fusion is

$$X = {\sum }_{i = 1}^{m} {W}_{m} {M}_{m} ∘$$

As a preferred scheme, when the vital sign extracted in step S4 is a heartbeat characteristic parameter, step S2, subcarrier fusion is performed on the channel state information of the wireless signal to obtain a vital sign waveform signal, which specifically includes the following steps:

K21, downsampling the channel state signal of the Wi-Fi signal to obtain downsampled channel state information. The sampling rate can be reduced as much as possible under the condition that the observed results can be satisfied, so that the calculation amount can be reduced and the real-time performance of the system can be improved. As a preferred scheme, the sampling rate can be reduced to 8 Hz, which can meet the requirements of wavelet transform.

K22, unwrapping the channel state information to obtain a preprocessed signal.

K23, performing subcarrier fusion processing on the preprocessed signal, performing frequency domain analysis, and outputting a heartbeat characteristic waveform signal.

As a preferred scheme, when the wireless signal is a 2.4G Wi-Fi signal, the frequency range that the subcarrier may cover is 2401 MHz to 2483 MHz. In practical use, one of the subcarriers in the bandwidth of 20 MHz or 40 MHz is generally selected.

When the wireless signal is a 5G Wi-Fi signal, the frequency range that the subcarrier may cover is 5150 MHz to 5850 MHz. In practical use, one of the subcarriers in the bandwidth of 20 MHz, 40 MHz or 80 MHz is generally selected. The higher the frequency, the shorter the wavelength of Wi-Fi signal, which is more sensitive to respiratory and heartbeat characteristics. Therefore, choosing the frequency range from 5750 MHz to 5850 MHz can achieve better detection effect.

### Embodiment 2

The difference between Embodiment 2 and Embodiment 1 is that the wireless signal used is not a Wi-Fi signal, but a millimeter-wave radar signal, and the frequency F of the millimeter-wave radar signal includes: 23 GHz≤F≤28 GHz, 60 GHz≤F≤65 GHz, 76 GHz≤F≤81 GHz. The phase of Wi-Fi signal is random. When the Wi-Fi signal returned by the measured object is received, the actual meaning of the reflected signal can not be known. Therefore, it is necessary to take the transmitted Wi-Fi signal as a reference signal, and calculate the phase difference between the reflected signal and the reference signal. Millimeter-wave radar signals can distinguish and identify very small targets, and can identify multiple targets at the same time. The phase information of millimeter-wave radar can directly reflect the micro motion characteristics of the reflecting surface, so it is not necessary to additionally calculate the phase difference in millimeter wave radar system.

As a preferred scheme, when the wireless signal is a millimeter wave radar signal, step S1 specifically includes the following steps:
S11, outputting a millimeter wave radar signal to a measured target, and taking the millimeter wave radar signal at the transmitting time as a reference signal;
S12, the measured target reflects the millimeter wave radar signal to form an echo signal, and the echo signal and the reference signal are demodulated to generate an intermediate frequency signal;
S13, performing ADC sampling and FFT transformation on the intermediate frequency signal in turn to obtain the distance information and the phase information of the measured target;
S14, the phase information of the millimeter wave radar signal is used as the channel state information of the millimeter wave radar signal.

As a preferred scheme of the present invention, the specific steps of step S11 are as follows: the controller controls the RF front-end to generate the required millimeter-wave radar waveform and transmit it, and stores the millimeter-wave radar signal at the transmitting time as the reference signal of the receiving end, and what is needed in this embodiment is the FMCW radar signal; In step S12, the RF front-end receives the echo signal of the millimeter-wave radar signal after passing through the reflecting surface (the measured target), and demodulates it with the reference signal to generate an intermediate frequency signal (IF).

In step S13, the obtained intermediate frequency signal includes the signal of the reflector. After sampling the intermediate frequency signal by ADC, the distance information and phase information of the reflector are obtained by FFT. Distance information is to get the distance between the reflector and the radar through different frequency points of FFT results; the phase information refers to the phase of 1D-FFT, and the phase information of 1D-FFT can reflect the slight change of the reflecting surface. The phase information of millimeter wave radar signal is regarded as the channel state information of millimeter wave radar signal. For millimeter-wave radar signals, the phase information itself carries the vital sign waveform signal. In step S2, the vital sign waveform signal can be directly extracted from the phase information of millimeter-wave radar signals by phase unwrapping. The method of phase unwrapping is the same as that in step S21 in Embodiment 1, and the details are not repeated here. Subsequent steps S3 and S4 are the same as the method in Embodiment 1, and will not be described here.

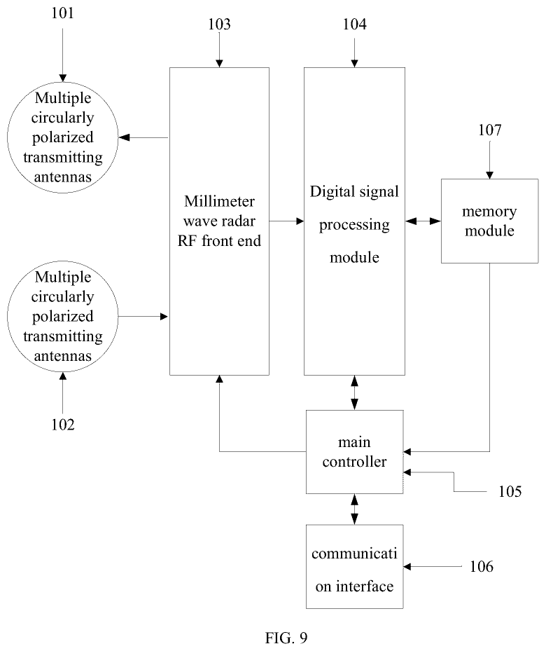

The schematic diagram of radar system data acquisition process is shown in FIG. 9. The system mainly includes millimeter wave radar RF front end, digital signal processing module, main controller, storage module and communication interface. The function of millimeter wave radar RF front-end is to generate and transmit millimeter wave radar signals under the control of the main controller, and receive radar echo signals, and get intermediate frequency signals according to the echo signals and reference signals (RF front-end is equivalent to the integration of wireless signal generating equipment and wireless signal receiving equipment). The function of the digital signal processing module is to sample the millimeter wave radar signal by ADC, then carry out FFT calculation and filtering, etc., and calculate the distance information, phase information, speed information and angle information. The storage module is used for storing the program and data of the detection system. The communication interface is the communication interface between the radar system and the automotive electronic system, which receives the instructions issued by the automotive electronic system and sends the data of the radar system to the automotive electronic system.

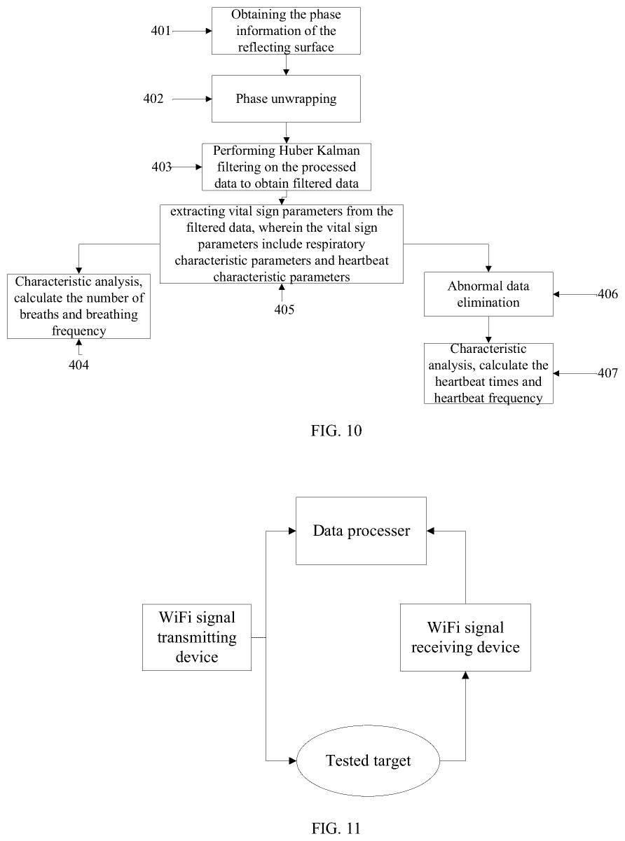

FIG. 10 is the flow chart of the vital sign detection software. After obtaining the echo signal and then calculating the phase information, directly unwrapping the phase information can directly obtain the vital sign waveform signal. By Huber-Kalman filtering the waveform signal of vital signs, the parameters of vital signs can be extracted, and the detection of vital signs includes two parameters: respiration and heartbeat. The phase information of radar echo signal reflects the micro motion characteristics of the target. Because the wavelength of millimeter wave is very short, the phase information can detect the micro motion characteristics of a few tenths of a millimeter, which can be used to detect breathing and heartbeat. Because the frequencies of respiratory and heartbeat are different, the characteristics of respiratory and heartbeat are separated after frequency domain analysis, and the number of respiratory and heartbeat is determined respectively (or the respiratory frequency and heartbeat frequency are determined separately). In the scene inside the car, the bumps of the car and the body movements of the people inside the car will bring different degrees of errors, which will affect the measurement results.

Wherein, the method of phase unwrapping and extracting vital parameters by Huber-Kalman filtering is the same as that of Embodiment 1 (steps S3-S4 in FIG. 1), and respiratory characteristic parameters and heartbeat characteristic parameters reflecting vital signs can be obtained, and will not be described here.

### Embodiment 3

Based on the same idea, Embodiment 3 gives a contactless breathing or heartbeat detection system, including a Wi-Fi signal transmitting device, a Wi-Fi signal receiving device and a data processor. The structural diagram of a contactless breathing or heartbeat detection system is shown in FIG. 11.

The Wi-Fi signal transmitting device outputs a Wi-Fi signal to the measured target; The Wi-Fi signal receiving device receives the Wi-Fi signal reflected by the measured target; The data processor generates a channel state signal of the Wi-Fi signal according to the Wi-Fi signal output by the Wi-Fi signal transmitting device and the reflected Wi-Fi signal received from the receiving antenna of the Wi-Fi signal, and performs subcarrier fusion on the channel state signal of the Wi-Fi signal to obtain a vital sign waveform signal;

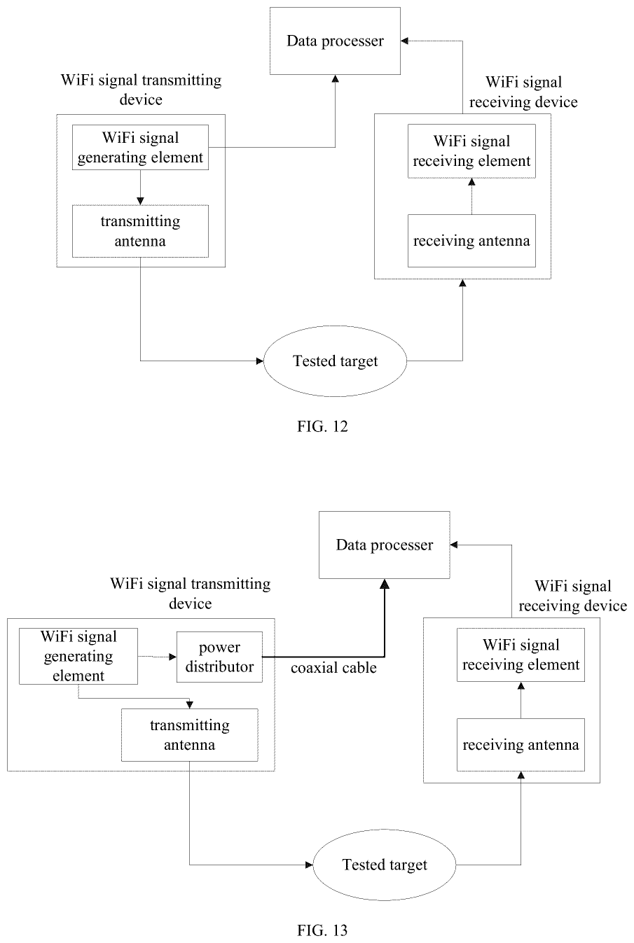

The data processor also filters the vital sign waveform signal based on Huber-Kalman filtering algorithm to obtain the filtered vital sign waveform signal, and extracts vital sign parameters from the filtered vital sign waveform signal, including respiratory characteristic parameters and heartbeat characteristic parameters; Huber-Kalman filtering algorithm uses Huber objective function to fuse the first norm and the second norm in Kalman function. Further, the Wi-Fi signal transmitting device includes a Wi-Fi signal generating element and a transmitting antenna, and the structural diagram of a contactless breathing or heartbeat detection system including the Wi-Fi signal generating element, the transmitting antenna, the Wi-Fi signal receiving device and the receiving antenna is shown in FIG. 12.

The transmitting antenna and the receiving antenna are circularly polarized, and the polarization directions of the transmitting antenna and the receiving antenna are opposite. If the transmitting antenna is a left-handed circularly polarized antenna, the receiving antenna is a right-handed circularly polarized antenna (or the transmitting antenna is a right-handed circularly polarized antenna, and the receiving antenna is a left-handed circularly polarized antenna). By using the circularly polarized antenna to suppress multipath interference, the direct signal and even reflected signal between the two antennas can be effectively suppressed, so that the signal received by the Wi-Fi signal receiving antenna is mainly a signal that has been reflected once, and the first reflected signal is a signal that is reflected from the measured target.

In addition, the system also includes a power distributor, and the contactless breathing or heartbeat detection system including the power distributor is shown in FIG. 13. The Wi-Fi signal generating element outputs the generated Wi-Fi signal to the power distributor, and the power distributor outputs the received Wi-Fi signal to the transmitting antenna and simultaneously outputs the Wi-Fi signal to the data processor through the coaxial cable; The data processor generates a channel state signal of the Wi-Fi signal according to the Wi-Fi signal received from the coaxial cable and the Wi-Fi signal reflected by the human body received from the receiving device of the Wi-Fi signal.

As a preferred scheme, the clock signal on which the Wi-Fi signal generating device generates the Wi-Fi signal is the same as that of the data processor. It avoids the error caused by the unsynchronized clocks of all parts of the system during signal processing, and increases the stability in signal processing.

The above is only the preferred embodiment of the invention, and it is not used to limit the invention. Any modification, equivalent substitution and improvement made within the spirit and principle of the invention should be included in the protection scope of the invention.

1. A contactless breathing or heartbeat detection method, including the following steps:
S1, acquiring channel state information of the wireless signal according to the output wireless signal and the wireless signal reflected by the measured target;
S2, extracting a vital sign waveform signal from the channel state information of the wireless signal;
S3, filtering the vital sign waveform signal based on a Huber-Kalman filtering algorithm to obtain a filtered vital sign waveform signal, wherein the Huber-Kalman filtering algorithm uses a Huber objective function to update a formula of the Kalman filtering algorithm;
S4, extracting respiratory characteristic parameters and/or heartbeat characteristic parameters from the filtered vital sign waveform signals.

2. A contactless breathing or heartbeat detection method as claimed in claim 1, wherein in step S3, the Huber-Kalman filtering algorithm uses the Huber objective function to update the formula of the Kalman filtering algorithm, specifically including the following steps:
the iterative calculation process of Kalman equation is modified by Huber objective function, and the input vital sign waveform signal is filtered;
Huber objective function divides the error into two parts, including big error and small error, big error refers to the error value that deviates from the real value and is greater than the big error threshold, and small error refers to the error value that fluctuates within the small error threshold based on the real value;
the iterative calculation means that the optimal estimated value at the current moment is determined by the optimal estimated value calculated according to Kalman update equation at the previous moment and the observed value calculated according to Huber objective function at the current moment.

3. A contactless breathing or heartbeat detection method as claimed in claim 2, wherein in step S3, the prediction equation based on Huber objective function is expressed as:
$${\hat{x}}_{k}^{-} = A {\hat{x}}_{k - 1} + B {u}_{k - 1}$$
$${ρ}_{a}^{-} ( {e}_{k + 1}^{-} ) = \{ \begin{matrix}{A}^{2} {ρ}_{a} ( {e}_{k} ) + \frac{Q}{2} & if 0 \leq {ρ}_{a} ( {e}_{k} ) \leq \frac{{a}^{2}}{2} \\ \vert A \vert ( {ρ}_{a} ( {e}_{k} ) + \frac{{a}^{2}}{2} ) - \frac{{a}^{2}}{2} & if {ρ}_{a} ( {e}_{k} ) > \frac{{a}^{2}}{2}\end{matrix}$$
Kalman update equation is:
$${K}_{k} = \{ \begin{matrix}\frac{2 {ρ}_{a}^{-} ( {e}_{k}^{-} ) H}{2 {ρ}_{a}^{-} ( {e}_{k}^{-} ) {H}^{2} + R} & if 0 \leq {ρ}_{a}^{-} ( {e}_{k}^{-} ) \leq \frac{{a}^{2}}{2} \\ \frac{1}{H} & if {ρ}_{a}^{-} ( {e}_{k}^{-} ) > \frac{{a}^{2}}{2}\end{matrix}$$
$${\hat{x}}_{k} = {\hat{x}}_{k}^{-} + {K}_{k} ( {z}_{k} - H {\hat{x}}_{k}^{-} )$$
$${ρ}_{a} ( {e}_{k} ) = \{ \begin{matrix}{ρ}_{a}^{-} ( {e}_{k}^{-} ) ( 1 + {K}_{k}^{2} {H}^{2} - 2 {K}_{k} H ) + \frac{1}{2} {K}_{k}^{2} R & if 0 \leq {ρ}_{a}^{-} ( {e}_{k}^{-} ) \leq \frac{{a}^{2}}{1 - {K}_{k} H} - \frac{{a}^{2}}{2} \\ \vert 1 - {K}_{k} H \vert ( {ρ}_{a}^{-} ( {e}_{k}^{-} ) + \frac{{a}^{2}}{2} ) - \frac{{a}^{2}}{2} & if {ρ}_{a}^{-} ( {e}_{k}^{-} ) > \frac{{a}^{2}}{1 - {K}_{k} H} - \frac{{a}^{2}}{2}\end{matrix}$$
where k represents the k th moment; a represents the threshold between a big error and a small error; \hat{x}ₖ⁻ represents the predicted value at moment k, and \hat{x}ₖ₋₁ represents the optimal estimated value at moment k−1; zₖ is the input data; uₖ₋₁ represents the random noise in the state transition process; vₖ represents measurement noise; Q represents the covariance of process noise; R represents the measurement noise covariance; A represents the state transition coefficient; B represents the control input coefficient; H represents the measurement coefficient; eₖ stands for posterior error; eₖ⁻ represents prior error; ρₐ⁻ (eₖ⁻) represents a prior error function; ρₐ (eₖ) represents a posterior error function; Kₖ stands for Kalman gain.

4. A contactless breathing or heartbeat detection method as claimed in claim 3, wherein step S3 further includes detecting the environmental noise level in real time, and adjusting the threshold between the big error and the small error according to the environmental noise level.

5. A contactless breathing or heartbeat detection method as claimed in claim 1, wherein step S4 specifically includes the following steps:
A41, segmenting the filtered vital sign waveform signal according to the time window to obtain a vital sign waveform;
A42, extracting the time interval between the peaks of the vital sign waveform, and determining the frequency of breathing or heartbeat of the tested human body according to the time interval between the peaks.

6. A contactless breathing or heartbeat detection method as claimed in claim 1, wherein step S4 specifically includes the following steps:
B41, segmenting the filtered vital sign waveform signal according to the time window to obtain a vital sign waveform;
B42, performing frequency domain analysis on the vital sign waveform to obtain the frequency spectrum characteristics of the vital sign waveform;
B43, performing low-pass filtering on the spectrum characteristics of the vital sign waveform to obtain the breathing frequency of the tested human body, and/or performing high-pass filtering on the spectrum characteristics of the vital sign waveform to obtain the heartbeat frequency of the tested human body.

7. A contactless breathing or heartbeat detection method as claimed in claim 1, wherein, when the wireless signal is a millimeter wave radar signal, step S1 specifically includes the following steps:
S11, outputting a millimeter wave radar signal to a measured target, and taking the millimeter-wave radar signal at the transmitting time as a reference signal;
S12, the measured target reflects the millimeter wave radar signal to form an echo signal, and the echo signal and the reference signal are demodulated to generate an intermediate frequency signal;
S13, performing ADC sampling and FFT transformation on the intermediate frequency signal in turn to obtain the distance information and the phase information of the measured target;
S14, the phase information of the millimeter wave radar signal is used as the channel state information of the millimeter wave radar signal.

8. A contactless breathing or heartbeat detection method as claimed in claim 7, wherein, the range of the frequency f of the millimeter wave radar signal includes: 23 GHz≤F≤28 GHz, 60 GHz≤F≤65 GHz and 76 GHz≤F≤81 GHz.

9. A contactless breathing or heartbeat detection method as claimed in claim 1, wherein, when the wireless signal is a Wi-Fi signal, step S1 specifically includes the following steps:
C11, outputting the wireless signal to the measured target, and simultaneously using the wireless signal as a reference signal, and transmitting the wireless signal in a wired way;
C12: the measured object reflects the wireless signal to form a reflected wireless signal, and the reflected wireless signal is differentiated from the reference signal to obtain the phase difference information of the wireless signal, which is used as the channel state information of the wireless signal.

10. A contactless breathing or heartbeat detection method as claimed in claim 9, wherein, step S2 specifically includes the following steps:
S21, unwrapping the phase information to obtain a preprocessed signal;
S22, performing subcarrier fusion processing on the preprocessed signal to output a respiratory characteristic waveform signal.

11. A contactless breathing or heartbeat detection method as claimed in claim 10, wherein, when the wireless signal is a Wi-Fi signal in 2.4G band, the frequency bandwidth of the channel state information is 20 MHz or 40 MHz, and the frequency range of the subcarrier signal of the channel state information is 2401 MHz to 2483 MHz;
when the wireless signal is a Wi-Fi signal in 5G band, the frequency bandwidth of the channel state information is 20 MHz, 40 MHz or 80 MHz, and the frequency range of the subcarrier signal of the channel state information is 5150 MHz to 5850 MHz.

12. A contactless breathing or heartbeat detection system, wherein comprises a wireless signal transmitting device, a wireless signal receiving device and a data processor,
the wireless signal transmitting device outputs a wireless signal to a measured target;
the wireless signal receiving device receives the wireless signal reflected by the measured target;
the data processor executes contactless vital sign detection method according to claim 1 according to the wireless signal output by the wireless signal transmitter and the wireless signal reflected by the measured target, and calculates the respiratory characteristic parameters and/or heartbeat characteristic parameters of the measured target.

13. A contactless breathing or heartbeat detection system, as claimed in claim 12, wherein the wireless signal transmitting device comprises a wireless signal generating element and a transmitting antenna, and the wireless signal receiving device comprises a receiving antenna and a wireless signal receiving element;
the wireless signal generating element radiates the generated wireless signal to the measured target through the transmitting antenna;
the wireless signal receiving element receives the wireless signal reflected by the measured target through the receiving antenna;
the transmitting antenna and the receiving antenna are circularly polarized, and the polarization directions of the transmitting antenna and the receiving antenna are opposite.

14. A contactless breathing or heartbeat detection system, as claimed in claim 12, wherein the clock signal on which the wireless signal generating element generates the wireless signal is the same as the clock signal on which the data processor receives the wireless signal reflected by the measured object.

15. A contactless breathing or heartbeat detection system, as claimed in claim 14, wherein when the wireless signal is a Wi-Fi signal, the system further comprises a power distributor,
the Wi-Fi signal generating element outputs the generated Wi-Fi signal to a power distributor,
the power distributor outputs the received Wi-Fi signal to the transmitting antenna and simultaneously outputs the Wi-Fi signal to the data processor through the coaxial cable;
the data processor generates the channel state information of the Wi-Fi signal according to the Wi-Fi signal received from the coaxial cable and the Wi-Fi signal reflected by the measured object.
